(19) United States
(12) Patent Application Publication
(10) Pub. No.: US 2005/0159809 A1
(43) Pub. Date: Jul. 21, 2005

(54) Implantable medical devices for treating or preventing restenosis

(75) Inventors: Ayala Hezi-Yamit, Windsor, CA (US); Sabeena Singh, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/009,863

(22) Filed: Dec. 10, 2004

Related U.S. Application Data
(60) Provisional application No. 60/538,189, filed on Jan. 21, 2004.

(51) Int. Cl. A61F 2/06

(52) U.S. Cl. 623/1.42

(57) ABSTRACT

Implantable medical devices having anti-restenotic antioxidants are disclosed. The anti-restenotic medical devices include stents and vascular grafts. Intravascular stents are preferred medical devices. The preferred anti-restenotic antioxidant is probucol. The medical devices can have coatings that include a polymer matrix. Related methods of treating or inhibiting restenosis using the Implantable medical devices are also disclosed.

### RELATED APPLICATIONS

This application claims the benefit of U.S. Provisional Patent Application 60/538,189 filed Jan. 21, 2004.

### FIELD OF THE INVENTION

The present invention relates to implantable medical devices provided having anti-restenotic coatings. Specifically, the present invention provides vascular stents having coatings releasing lipid soluble antioxidants wherein the antioxidants have anti-restenotic properties.

### BACKGROUND OF THE INVENTION

The implantation of medical devices has become a relatively common technique for treating a variety of medical or disease conditions within a patient's body. Depending upon the conditions being treated, today's medical implants can be positioned within specific portions of a patient's body where they can provide beneficial functions for periods of time ranging from days to years. A wide variety of medical devices can be considered implants for purposes of the present invention. Such medical devices can include structural implants such as stents and internal scaffolding for vascular use, replacement parts such as vascular grafts, or in-dwelling devices such as probes, catheters and microparticles for monitoring, measuring and modifying biological activities within a patient's cardiovascular system. Other types of medical implants for treating different types of medical or disease conditions can include in-dwelling access devices or ports, valves, plates, barriers, supports, shunts, discs, and joints, to name a few.

One form of cardiovascular disease, commonly referred to as atherosclerosis, remains a leading cause of death in developed countries. Atherosclerosis is a disease that results in the narrowing, or stenosis, of blood vessels which can lead to heart attack or stroke if the narrowing progresses to the point of blocking blood flow through the narrowed blood vessels forming the coronary arteries. Cardiovascular disease caused by stenotic or narrowed coronary arteries is commonly treated using either a coronary artery by-pass graft (CABG) around the blockage, or a less invasive procedure called angioplasty where a balloon catheter is inserted into the blocked coronary artery and advanced until the vascular stenosis is reached by the advancing balloon. The balloon is then inflated to deform the stenosis open, restoring blood flow.

However, angioplasty or balloon catheterization can result in internal vascular injury which may ultimately lead to reformation of narrowing vascular deposits within the previously opened artery. This biological process whereby a previously opened artery becomes re-occluded is called restenosis. One angioplasty variation designed to reduce the possibility of restenosis includes the subsequent step of arterial stent deployment within the stenotic blockage opened by the expanded balloon. After arterial patency has been restored by expanding the angioplasty balloon to deform the stenotic lesion open, the balloon is deflated and a vascular stent is inserted into the tubular bore or vessel lumen across the stenosis site. The catheter is then removed from the coronary artery lumen and the deployed stent remains implanted across the opened stenosis to prevent the newly opened artery from constricting spontaneously or narrowing in response to the internal vascular injury resulting from the angioplasty procedure itself. However, it has been found that in some cases of angioplasty and angioplasty followed by stent deployment restenosis may still occur.

Treating restenosis generally requires additional, more invasive, procedures including CABG. Consequently, methods for preventing restenosis, or for treating incipient forms of restenosis, are being aggressively pursued. One promising method for preventing restenosis is the administration of medicaments that block the local invasion or activation of monocytes (white blood cells that respond to injury or infection). Monocytes secrete growth factors within the blood vessel at the restenosis site that can trigger vascular smooth muscle cell (VSMC) proliferation and migration causing thickening of the vessel wall and subsequent narrowing of the artery. Metabolic inhibitors such as anti-neoplastic agents are currently being investigated as potential anti-restenotic compounds for such purposes. However, the toxicity associated with the systemic administration of known metabolic inhibitors has more recently stimulated development of in situ or site-specific drug delivery designed to place the anti-restenotic compounds directly at the target site within the potential restenotic lesion rather than generally administering much larger, potentially toxic doses to the patient.

For example, one particular site-specific drug delivery technique known in the art employs the use of vascular stents coated with anti-restenotic drugs. These stents have been particularly useful because they not only provide the mechanical structure to maintain the patency or openness of the damaged vessel, but they also release the anti-restenotic agents directly into the surrounding tissue. This site specific delivery allows clinically effective drug concentrations to be achieved locally at the stenotic site without subjecting the patient to the side effects that may be associated with systemic drug delivery. Moreover, localized or site-specific delivery of anti-restenotic drugs eliminates the need for more complex specific cell targeting technologies intended to accomplish similar purposes.

It has been recognized that macrophage-derived foam-cell formation may be dependent on the oxidative modification of low density lipoprotein (LDL) and its subsequent uptake via a scavenger receptor-mediated pathway (Steiberg, D. Parthasarathy, S. Carew, T. E. Khoo, J. C. and Witzum, J. L. (1989) N. Engl. J. Med. 320, 915-924). Moreover, LDL oxidation breakdown products have been associated with VSMC and macrophage chemotaxis. Therefore, compounds that specifically inhibit LDL uptake and oxidation (e.g. lipid soluble antioxidants) may attenuate this process and reduce or prevent restenosis following angioplasty. In 1992 studies were conducted at the William Harvey Research Institute, London, UK that examined the effects of one such lipid soluble-antioxidant, probucol, on balloon-injury induced neointimal thickening and macrophage accumulation in cholesterol-fed rabbits (Ferns, G. A. A., Forster, L. Stewart-Lee, A., Konneh, M. Nourooz-Zadeh, J. and Anggard, E. E. (1992). Probucol inhibits neointimal thickening and macrophage accumulation after balloon injury in cholesterol-fed rabbit. Proc. Natl. Acad. Sci. USA: Vol. 89, pp. 11312-11316). In this study juvenile New Zealand White rabbits (3-6 months in age) were fed a commercial rabbit chow for one week and then divided into three research groups. One group received a high cholesterol diet alone and a second group was fed high cholesterol rabbit food plus 1% probucol (Merrell-Dow, now Aventis S.A.), a third group served as control and received normal rabbit chow. After one week the carotid arteries were de-endothelialized using a balloon catheter. After four weeks the animals were sacrificed and their carotid arteries were dissected and microscopically examined. The animals receiving probucol demonstrated lower macrophage content in the neointima compared to animals receiving high cholesterol feed but no probucol (P<0.001). Moreover, the absolute neointima thickness of the probucol fed group was also reduced relative to the cholesterol-only diet (P<0.05). Therefore, it was concluded that systemic prophylaxis with probucol could reduce neointimal thickening and macrophage accumulation (i.e. restenosis) following balloon angioplasty.

The exact mechanism of probucol's anti-restenosis activity is not well defined. However, it is believed that probucol may prevent macrophage activation and macrophage-derived foam cell formation thereby suppressing monokine release. The William Harvey Research Institute study used probucol systemically and the test animals were fed probucol prophylactically for seven days before angioplasty. While these studies provide intriguing clues to the potential of probucol as an anti-restenotic, the systemic use of probucol is not United States (US) Food and drug Administration (FDA) approved and is known to have systemic side effects. Specifically, its maker, Merrell-Dow (now Aventis S.A.), removed probucol from the market in 1995 after reports that probucol could disrupt the electrical impulses that guide the heart's rhythms. However, no testing has been done using probucol as an anti-restenotic deployed from a medical device such as a vascular stent. Generally, site-specific drug deployment differs significantly from systemic applications. Site specific applications are generally for shorter time periods and much lower drug concentrations when compared to systemic applications. Thus, the side effects associated with long-term systemic drug delivery are much less likely to occur with short-term site-specific drug delivery.

Therefore, there is a need for alternative approaches for delivering compounds showing promising anti-restenotic activity in animals that may have toxic side effects when used systemically. Consequently, it is an object of the present invention to provide vascular stents and stent coatings having anti-restenotic effective amounts of lipid-soluble antioxidants.

### SUMMARY OF THE INVENTION

In one embodiment of the present invention an implantable medical device having at least one anti-restenotic antioxidant.

In yet another embodiment the present invention is an implantable medical device selected from the group consisting of vascular stents, urethral stents, biliary stents and endovascular grafts.

In one embodiment of the present invention an implantable medical device is provided with a lipid soluble anti-restenotic antioxidant.

In another embodiment the lipid soluble anti-restenotic antioxidant is {[bis(3,5-di-tert-butyl-4-hydroxyphenyl)thio]propane} (probucol).

The present invention may also include implantable medical devices having coatings that include a polymer matrix wherein the polymer matrix is formed from at least one biocompatible polymer selected from the group consisting of polyurethanes, silicones, polyesters, polyolefins, polyisobutylene and ethylene-alphaolefin copolymers, acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, polyvinyl esters, copolymers of vinyl monomers, ethylene-methyl methacrylate copolymers, acrylonitrile-styrene copolymers, ABS resins, ethylene-vinyl acetate copolymers; polyamides, alkyd resins; polycarbonates, polyoxymethylenes, polyimides, polyethers, epoxy resins, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, phosphatidylcholine, fibrin and combinations thereof.

Also included within the scope of the present invention are methods for treating or inhibiting restenosis that include administering an anti-restenotic antioxidant to a specific site in a mammalian vessel subject to restenosis such that restenosis is treated or inhibited.

In one embodiment of the present invention the mammalian vessel subject to restenosis is the vessel lumen or adventitia and the administered anti-restenotic antioxidant is probucol.

### BRIEF DESCRIPTION OF THE DRAWINGS

FIG. 1 depicts a vascular stent used to deliver the anti-restenotic compounds of the present invention.

FIG. 2 depicts a balloon catheter assembly used for angioplasty and the site-specific delivery of stents to anatomical lumens at risk for restenosis.

FIG. 3 depicts the needle of an injection catheter in the retracted position (balloon deflated) according to the principles of the present invention where the shaft is mounted on an intravascular catheter.

FIGS. 4 and 5 illustrate use of the apparatus of FIG. 3 in delivering a substance into the adventitial tissue surrounding a blood vessel.

### DETAILED DESCRIPTION OF THE PRESENT INVENTION

The present invention provides stents having that provide anti-restenotics directly to the cells at the site of stent implantation. Specifically, the present invention provides means for delivering antioxidant anti-restenotics to an arterial intima or adventitia either before, after or during a clinical procedure. In one embodiment of the present invention a vascular stent is provided with a coating comprising at least one antioxidant anti-restenotic. In an exemplary embodiment the present invention includes a stent having a coating that releases {[bis(3,5-di-tert-butyl-4-hydroxyphenyl)thio]propane} a potent lipid soluble antioxidant also known as probucol. Probucol is marketed by Aventis Pharma Canada as a systemic antihyperlipemic under the brand name Lorelco and is also sold under the generic names Bifenabid, Lesterol, Lurselle, Panesclerina and Superlipid; probucol is not available for systemic use in the U.S.

Probucol is a lipophilic compound that reduced serum cholesterol levels through a mechanism not entirely understood; however, recent studies suggest that probucol may interfere with low density lipoprotein (LDL) modification and prevent cholesterol uptake. Studies conducted in the early 1990s suggested that probucol may inhibit neointimal thickening and macrophage accumulation after balloon injury in cholesterol fed rabbits. Based on these studies it was proposed that probucol may be useful as an anti-restenotic. However, only the prophylactic benefits of systemically administered probucol were considered.

It is proposed, and not intended as a limitation, that probucol's anti-restenotic activity is related to suppression of macrophage activation and adhesion molecule expression. Moreover, it is also proposed that probucol prevents injured intima cells from expressing chemotactic agents that recruit macrophages and stimulate vascular smooth muscle cell (VSMC) proliferation. One possible mechanism may be probucol's ability to quench reactive oxygen species and inhibition of interleukin I)IL-1) secretion from lipopolysaccaride-stimulated macrophages (Akeson, A. L., Woods, C. W., Mosher, L. B., Thomas, C. E. and Jackson, R. L. (1991) Inhibition of IL-1 beta expression in THP-1 cells by probucol and tocopherol. Atherosclerosis 86(2-3): 261-70; Rao, G. N. and Beck, B. C. (1992) Active oxygen species stimulate vascular smooth muscle cell growth and proto-oncogene expression. Circ Res. 70(3): 593-9). Moreover, probucol inhibits oxidation of both LDL and beta-very low density lipoproteins (B-VLDL) and thus inhibit oxidized LDL-induced adhesion molecule expression and reduce IL-I's VSMC mitogen activity (Hara, S., Nagano, Y., Sasada, M. and Kita, T. (1992) Probucol pretreatment enhances the chemotaxis of mouse peritoneal macrophages. Arterioscler Thromb. 12(5): 593-600).

Based on these proposed mechanisms of action the present inventions will provide medical implants having anti-restenotic coatings that release anti-restenotic effective amounts of probcol and other antioxidants having mechanisms of action similar to those proposed herein. In another embodiment of the present invention a stent has a coating comprising probucol and at least one biocompatible polymer.

The stents used in accordance with the teachings of the present invention may be vascular stents, urethral stents, biliary stents, endovascular grafts or stents intended for use in other ducts and organ lumens. Vascular stents may be used in peripheral, neurological or coronary applications. The stents may be rigid expandable stents or pliable self-expanding stents. Any biocompatible material may be used to fabricate the stents of the present invention including, without limitation, metals or polymers. The stents of the present invention may also be bioresorbable.

The anti-restenotic antioxidant may be dissolved or suspended in any carrier compound that provides a stable composition that does not react adversely with the device to be coated or inactivate the anti-restenotic antioxidants of the present invention. A metallic stent is provided with a biologically active anti-restenotic antioxidant coating using any technique known to those skilled in the art of medical device manufacturing. Suitable non-limiting examples include impregnation, spraying, brushing, dipping and rolling. After the anti-restenotic antioxidant solution is applied to the stent it is dried leaving behind a stable anti-restenotic antioxidant delivering medical device. Drying techniques include, but are not limited to, heated forced air, cooled forced air, and vacuum drying or static evaporation. Moreover, the medical device, specifically a metallic vascular stent, can be fabricated having grooves or wells in its surface that serve as receptacles or reservoirs for the anti-restenotic antioxidant compositions of the present invention.

A titration process can determine the anti-restenotic effective amounts of antioxidants used in accordance with the teachings of the present invention. Titration is accomplished by preparing a series of stent sets. Each stent set will be coated, or contain different dosages of the anti-restenotic antioxidant selected. The highest concentration used will be partially based on the known toxicology of the compound. The maximum amount of drug delivered by the stents made in accordance with the teaching of the present invention will fall below known toxic levels. Each stent set will be tested in vivo using the preferred animal model. The dosage selected for further studies will be the minimum dose required to achieve the desired clinical outcome. In the case of the present invention, the desired clinical outcome is defined as the inhibition of vascular re-occlusion, or restenosis. Generally, and not intended as a limitation, an anti-restenotic effective amount of the antioxidants of the present invention will range between about 0.5 ng to 1.0 mg depending on the anti-restenotic antioxidant used and the delivery platform selected.

In addition to the anti-restenotic antioxidant selected, treatment efficacy may also be affected by factors including dosage, route of delivery and the extent of the disease process (treatment area). An effective amount of an anti-restenotic antioxidant composition can be ascertained using methods known to those having ordinary skill in the art of medicinal chemistry and pharmacology. First the toxicological profile for a given anti-restenotic antioxidant composition is established using standard laboratory methods. For example, the candidate anti-restenotic antioxidant composition is tested at various concentrations in vitro using cell culture systems in order to determine cytotoxicity. Once a non-toxic, or minimally toxic, concentration range is established, the anti-restenotic antioxidant composition is tested throughout that range in vivo using a suitable animal model. After establishing the in vitro and in vivo toxicological profile for the anti-restenotic antioxidant compound, it is tested in vitro to ascertain if the compound retains anti-restenotic activity at the non-toxic, or minimally toxic ranges established.

Finally, the candidate anti-restenotic antioxidant composition is administered to humans in accordance with either approved Food and Drug Administration (FDA) clinical trial protocols, or protocol approved by Institutional Review Boards (IRB) having authority to recommend and approve human clinical trials for minimally invasive procedures. Treatment areas are selected using angiographic techniques or other suitable methods known to those having ordinary skill in the art of intervention cardiology. The candidate anti-restenotic antioxidant composition is then applied to the selected treatment areas using a range of doses. Preferably, the optimum dosages will be the highest non-toxic, or minimally toxic concentration established for the anti-restenotic antioxidant composition being tested. Clinical follow-up will be conducted as required to monitor treatment efficacy and in vivo toxicity. Such intervals will be determined based on the clinical experience of the skilled practitioner and/or those established in the clinical trial protocols in collaboration with the investigator and the FDA or IRB supervising the study.

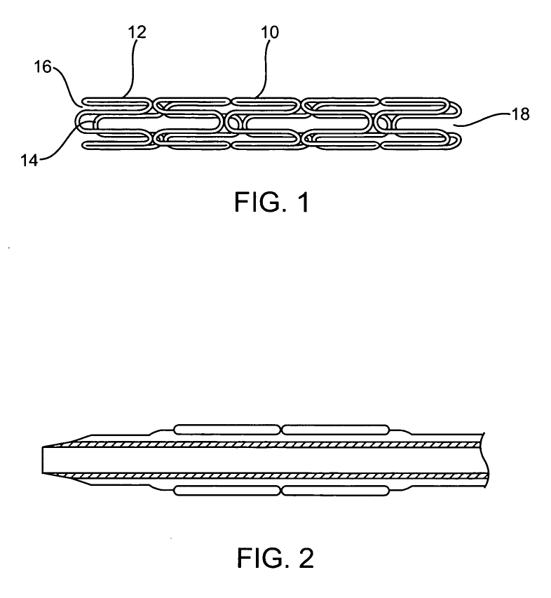

The anti-restenotic antioxidant therapy of the present invention can be administered directly to the treatment area using any number of techniques and/or medical devices. In one embodiment of the present invention the anti-restenotic antioxidant composition is applied to a vascular stent. The vascular stent can be of any composition or design. For example, the stent 10 (FIG. 1) may be a self-expanding stent or may be mechanically expanded using a balloon catheter FIG. 2. The stent 10 may be made from stainless steel, titanium alloys, nickel alloys or biocompatible polymers. Furthermore, the stent 10 may be polymeric or a metallic stent coated with at least one polymer. In other embodiments the delivery device is an aneurysm shield, a vascular graft or surgical patch. In yet other embodiments the anti-restenotic antioxidant therapy of the present invention is delivered using a porous or “weeping” catheter to deliver a anti-restenotic antioxidant containing hydrogel composition to the treatment area. Still other embodiments include microparticles delivered using a catheter or other intravascular or transmyocardial device.

In another embodiment an injection catheter can be used to deliver the anti-restenotic antioxidants of the present invention either directly into, or adjacent to, a vascular occlusion or a vasculature site at risk for developing restenosis (treatment area). As used herein, adjacent means a point in the vasculature either distal to, or proximal from a treatment area that is sufficiently close enough for the anti-restenotic composition to reach the treatment area at therapeutic levels. A vascular site at risk for developing restenosis is defined as a treatment area where a procedure is conducted that may potentially damage the luminal lining. Non-limiting examples of procedures that increase the risk of developing restenosis include angioplasty, stent deployment, vascular grafts, ablation therapy, and brachytherapy.

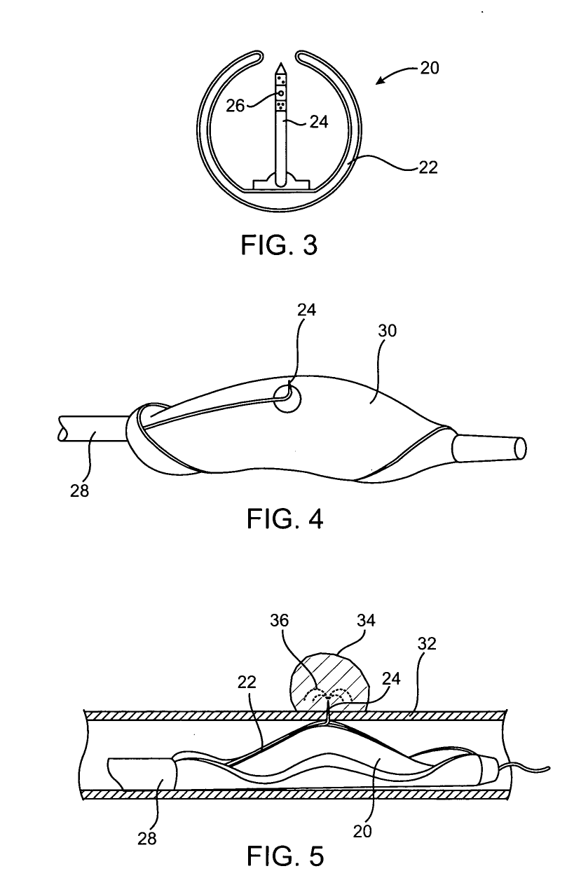

In one embodiment of the present invention an injection catheter as depicted in United States patent application publication No. 2002/0198512 A1, U.S. patent application Ser. No. 09/961,079 and U.S. Pat. No. 6,547,803 (specifically those portions describing adventitial delivery of pharmaceutically active compositions which are hereby incorporated herein by reference) can be used to administer the anti-restenotic antioxidants of the present invention directly to the adventia. FIGS. 3, 4 and 5 depict one such embodiment. FIG. 3 illustrates the C-shaped configuration of the catheter balloon 20 prior to inflation having the injection needle 24 nested therein and a balloon interior 22 connected to an inflation source (not shown) which permits the catheter body to be expanded as shown in FIG. 4. Needle 24 has an injection port 26 that transits the anti-restenotic antioxidant into the adventia from a proximal reservoir (not shown) located outside the patient.

FIG. 4 illustrates the inflated balloon 30 attached to the catheter body 28 and injection needle 24 capable of penetrating the adventia. FIG. 5 depicts deployment of the anti-restenotic antioxidant of the present invention directly into the adventia 34. The injection needle 24 penetrates the blood vessel wall 32 as balloon 20 is inflated and injects the anti-restenotic antioxidant 36 into the tissue.

The medical device can be made of virtually any biocompatible material having physical properties suitable for the design. For example, tantalum, stainless steel and nitinol have been proven suitable for many medical devices and could be used in the present invention. Also, medical devices made with biostable or bioabsorbable polymers can be used in accordance with the teachings of the present invention. Although the medical device surface should be clean and free from contaminants that may be introduced during manufacturing, the medical device surface requires no particular surface treatment in order to retain the coating applied in the present invention. Both surfaces (inner 14 and outer 12 of stent 10, or top and bottom depending on the medical devices' configuration) of the medical device may be provided with the coating according to the present invention.

In order to provide the coated medical device according to the present invention, a solution which includes a solvent, a polymer dissolved in the solvent and a anti-restenotic antioxidant composition dispersed in the solvent is first prepared. It is important to choose a solvent, a polymer and a therapeutic substance that are mutually compatible. It is desirable that the solvent is capable of placing the polymer into solution at the concentration desired in the solution. It is also desirable that the solvent and polymer chosen do not chemically alter the anti-restenotic antioxidant's therapeutic character. However, the anti-restenotic antioxidant composition only needs to be dispersed throughout the solvent; it may be a true solution or dispersed as fine particles in the solvent. Although the term “solution or mixture” may be used herein for convenience, it is not intended as a limitation and the although the solubility of the drug (anti-restenotic antioxidant) and polymer(s) may be closely match, it is not essential and a true homogenous solution be obtained. In fact, in some embodiments of the present invention a gradient of drug-polymer(s) may be desired. The polymer/drug mixture is applied to the medical device and the solvent is allowed to evaporate leaving a coating on the medical device comprising the polymer(s) and the anti-restenotic antioxidant composition.

Typically, the solution can be applied to the medical device by either spraying the solution onto the medical device or immersing the medical device in the solution. Whether one chooses application by immersion or application by spraying depends principally on the viscosity and surface tension of the solution, however, it has been found that spraying in a fine spray such as that available from an airbrush will provide a coating with the greatest uniformity and will provide the greatest control over the amount of coating material to be applied to the medical device. In either a coating applied by spraying or by immersion, multiple application steps are generally desirable to provide improved coating uniformity and improved control over the amount of anti-restenotic antioxidant composition to be applied to the medical device. The total thickness of the polymeric coating will range from approximately 1 micron to about 20 microns or greater. In one embodiment of the present invention the anti-restenotic antioxidant composition is contained within a base coat, and a top coat is applied over the anti-restenotic antioxidant containing base coat to control release of the anti-restenotic antioxidant into the tissue.

The polymer chosen should be a polymer that is biocompatible and minimizes irritation to the vessel wall when the medical device is implanted. The polymer may be either a biostable or a bioabsorbable polymer depending on the desired rate of release or the desired degree of polymer stability. Bioabsorbable polymers that could be used include poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid.

Also, biostable polymers with a relatively low chronic tissue response such as polyurethanes, silicones, and polyesters could be used and other polymers could also be used if they can be dissolved and cured or polymerized on the medical device such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose and phosphatidylcholine (PC).

The polymer-to-anti-restenotic antioxidant composition ratio will depend on the efficacy of the polymer in securing the anti-restenotic antioxidant composition onto the medical device and the rate at which the coating is to release the anti-restenotic antioxidant composition to the tissue of the blood vessel. More polymer may be needed if it has relatively poor efficacy in retaining the anti-restenotic antioxidant composition on the medical device and more polymer may be needed in order to provide an elution matrix that limits the elution of a very soluble anti-restenotic antioxidant composition. A wide ratio of therapeutic substance-to-polymer could therefore be appropriate and could range from about 0.1% to 99% by weight of therapeutic substance-to-polymer.

In one embodiment of the present invention a vascular stent as depicted in FIG. 1 is coated with anti-restenotic antioxidant using a two-layer biologically stable polymeric matrix comprised of a base layer and an outer layer. Stent 10 has a generally cylindrical shape and an outer surface 12, an inner surface 14, a first open end 16, a second open end 18 and wherein the outer and inner surfaces 12, 14 are adapted to deliver an anti-restenotic effective amount of at least one anti-restenotic antioxidant in accordance with the teachings of the present invention. Briefly, a polymer base layer comprising a solution of ethylene-co-vinylacetate and polybutylmethacrylate is applied to stent 10 such that the outer surface 12 is coated with polymer. In another embodiment both the inner surface 14 and outer surface 12 of stent 10 are provided with polymer base layers. The anti-restenotic antioxidant or mixture thereof is incorporated into the base layer. Next, an outer layer comprising only polybutylmethacrylate is applied to stent's 10 outer layer 14 that has been previous provided with a base layer. In another embodiment both the inner surface 14 and outer surface 12 of stent 10 are provided with polymer outer layers.

The thickness of the polybutylmethacrylate outer layer determines the rate at which the anti-restenotic antioxidant elutes from the base coat by acting as a diffusion barrier. The ethylene-co-vinylacetate, polybutylmethacrylate and anti-restenotic antioxidant solution may be incorporated into or onto a medical device in a number of ways. In one embodiment of the present invention the anti-restenotic antioxidant/polymer solution is sprayed onto the stent 10 and then allowed to dry. In another embodiment, the solution may be electrically charged to one polarity and the stent 10 electrically changed to the opposite polarity. In this manner, the anti-restenotic antioxidant/polymer solution and stent will be attracted to one another thus reducing waste and providing more control over the coating thickness.

In another embodiment of the present invention the anti-restenotic antioxidant is probucol and the polymer is bioresorbable. The bioresorbable polymer-anti-restenotic antioxidant blends of the present invention can be designed such that the polymer absorption rate controls drug release. In one embodiment of the present invention a polycaprolactone-anti-restenotic antioxidant blend is prepared. A stent 10 is then stably coated with the polycaprolactone-probucol blend wherein the stent coating has a thickness of between approximately 0.1 μm to approximately 100 μm The polymer coating thickness determines the total amount of probucol delivered and the polymer's absorption rate determines the administrate rate.

Using the teachings herein it is possible for one of ordinary skill in the part of polymer chemistry to design coatings having a wide range of dosages and administration rates. Furthermore, drug delivery rates and concentrations can also be controlled using non-polymer containing coatings and techniques known to persons skilled in the art of medicinal chemistry and medical device manufacturing.

1. An implantable medical device comprising:
a coating having at least one anti-restenotic antioxidant.

2. The implantable medical device according to claim 1 further comprising a biocompatible polymer matrix.

3. The implantable medical device according to claim 1 or claim 2 wherein said medical device is selected from the group consisting of vascular stents, urethral stents, biliary stents and endovascular grafts.

4. The implantable medical device according to claim 3 wherein said anti-restenotic antioxidant is lipid soluble.

5. The implantable medical device according to claim 4 wherein said lipid soluble anti-restenotic antioxidant is {[bis(3,5-di-tert-butyl-4-hydroxyphenyl)thio]propane} (probucol).

6. The implantable medical device according to claim 4 or 5 wherein said polymer matrix comprises at least one biocompatible polymer selected from the group consisting of polyurethanes, silicones, polyesters, polyolefins, polyisobutylene and ethylene-alphaolefin copolymers, acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, polyvinyl esters, copolymers of vinyl monomers, ethylene-methyl methacrylate copolymers, acrylonitrile-styrene copolymers, ABS resins, ethylene-vinyl acetate copolymers; polyamides, alkyd resins; polycarbonates, polyoxymethylenes, polyimides, polyethers, epoxy resins, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, phosphatidylcholine, fibrin and combinations thereof.

7. A method for treating or inhibiting restenosis comprising:
administering an anti-restenotic antioxidant to a specific site in a mammalian vessel at subject to restenosis such that restenosis is treated or inhibited.

8. The method according to claim 7 wherein said specific site in said mammalian vessel subject to restenosis is the vessel lumen or adventitia.

9. The method according to claim 7 or 8 wherein said administered anti-restenotic antioxidant is probucol.

10. The method according to claim 7 wherein said anti-restenotic antioxidant is administered by means of an implantable medical device having a coating comprising said anti-restenotic antioxidant and a polymer matrix.
